(19) United States
(12) Patent Application Publication
(10) Pub. No.: US 2017/0151034 A1
(43) Pub. Date: Jun. 1, 2017

(54) DISPLAY CONTROL DEVICE, DISPLAY CONTROL METHOD, DISPLAY CONTROL SYSTEM, AND HEAD-MOUNTED DISPLAY

(71) Applicant: SONY CORPORATION, Tokyo (JP)

(72) Inventors: Kyoichiro ODA, Tokyo (JP); Takahito WAKEBAYASHI, Kanagawa (JP)

(73) Assignee: SONY CORPORATION, Tokyo (JP)

(21) Appl. No.: 15/325,754

(22) PCT Filed: Aug. 21, 2015
(86) PCT No.: PCT/JP2015/004199

§ 371 Date: Jan. 12, 2017

(30) Foreign Application Priority Data
Sep. 16, 2014 (JP) 2014-187583

(51) Int. Cl. A61B 90/00; A61B 1/00; A61B 1/04

(52) U.S. Cl. CPC A61B 90/37; A61B 1/04; A61B 1/00048

(57) ABSTRACT

Provided is a surgical display system. The circuitry (200) is configured to receive user identification information and determine a display (100A, 100B) setting for each of a plurality of head-mounted displays based on display setting information associated with the received user identification information. The circuitry is further configured to set the display settings of the plurality of head-mounted displays based on the determined display settings to display an image from a surgical imaging device.

### CROSS REFERENCE TO RELATED APPLICATIONS

This application claims the benefit of Japanese Priority Patent Application JP 2014-187583 filed Sep. 16, 2014, the entire contents of which are incorporated herein by reference.

### TECHNICAL FIELD

The present disclosure relates to a display control device, a display control method, a display control system, and a head-mounted display.

### BACKGROUND ART

As one type of wearable terminals that users wear and use, there is a head-mounted display (hereinafter referred to as an “HMD”). An HMD is a display device that is worn on the head of a user for use, and has been used recently not only as a display device for AV devices, computer games, or the like but also as a display device for a user to check information while working in a work environment.

For example, in the field of medicine, HMDs are used as display devices for projecting endoscopic videos. An operator wears an HMD and conducts surgery while viewing a video projected on the HMD. Up until now, endoscopic videos were generally displayed on a monitor installed near operators, and thus the operators had to move their lines of sight between the monitor and an affected site very often. By projecting endoscopic videos on an HMD, operators can check affected sites and the endoscopic videos displayed in a display unit of the HMD without turning their lines of sight often.

Here, when a plurality of users use HMDs in an operating room, display content to be displayed in display units of the HMDs and display settings differ according to roles and preferences of the users. A display setting of a display unit can normally be set each time the HMD is worn, but this is cumbersome. Thus, a technology in which display setting information is stored in advance, and when a password of a user who will use an HMD is identified, a display setting of the HMD is automatically performed based on the display setting information associated with the password has also been proposed (for example, PTL 1).

### CITATION LIST

### Patent Literature

PTL 1: JP 09-93513A

### SUMMARY

### Technical Problem

However, because a display setting is performed based on display setting information of a user stored in the HMD in the technology described in PTL1 above, an HMD of which a display setting is automatically performed is fixed for each user. In addition, when it is desired to make the same display setting of HMDs for a plurality of users, it is necessary to make the display setting for the respective HMDs.

Thus, an embodiment of the present disclosure proposes a novel and improved display control device, display control method, display control system, and head-mounted display which enable an easy display setting of a plurality of HMDs.

### Solution to Problem

According to an embodiment of the present disclosure, there is provided a surgical system, including a surgical imaging device configured to capture a surgical image;
a plurality of head-mounted displays; and circuitry configured to receive user identification information from each of the plurality of head-mounted displays, determine a display setting for each of the plurality head-mounted displays based on display setting information associated with the user identification information received from the respective one of the plurality of head-mounted displays, and set the display settings of the plurality of head-mounted displays based on the determined display settings to display the surgical image from the surgical imaging device.

According to another embodiment of the present disclosure, there is provided a surgical display system, including circuitry configured to receive user identification information, determine a display setting for each of a plurality of head-mounted displays based on display setting information associated with the received user identification information, and set the display settings of the plurality of head-mounted displays based on the determined display settings to display an image from a surgical imaging device.

According to another embodiment of the present disclosure, there is provided a method of a surgical display system for controlling display of a image, including receiving user identification information; determining, by circuitry of the surgical display system, a display setting for each of a plurality of head-mounted displays based on display setting information associated with the received user identification information; and setting, by the circuitry, the display settings of the plurality of head-mounted displays based on the determined display settings.

According to another embodiment of the present disclosure, there is provided a head-mounted display including circuitry configured to acquire at least user identification information; and output the acquired user identification information to a display control device.

### Advantageous Effects of Invention

According to an embodiment of the present disclosure described above, it is possible to easily perform respective display settings of a plurality of HMDs. It should be noted that the effects described above are not necessarily limited, and along with or instead of the effects, any effect that is desired to be introduced in the present specification or other effects that can be expected from the present specification may be exhibited.

### BRIEF DESCRIPTION OF DRAWINGS

FIG. 1 is a system configuration diagram illustrating a configuration example of an endoscope system according to a first embodiment of the present disclosure.

FIG. 2 is an illustrative diagram for describing an operation of a user at the time of a display setting of an HMD according to the embodiment.

FIG. 3 is a functional block diagram illustrating a functional configuration of an HMD and a processor unit which constitutes a display control system according to the embodiment.

FIG. 4 is an illustrative diagram for describing a direction of display of a video that is one type of display setting information of the HMD.

FIG. 5 is an illustrative diagram for describing disposition of videos which is one type of the display setting information of the HMD.

FIG. 6 is a flow chart showing a display process based on the display setting information of the display control system according to the embodiment.

FIG. 7 is an illustrative diagram illustrating an example in which a user name is displayed in the processor unit.

FIG. 8 is an illustrative diagram illustrating an example in which a user ID is displayed in the processor unit.

FIG. 9 is an illustrative diagram illustrating an example in which an image being displayed in the HMD is displayed in the processor unit.

FIG. 10 is an illustrative diagram illustrating a notification object displayed in an external display.

FIG. 11 is a functional block diagram illustrating a functional configuration of an HMD and a processor unit constituting a display control system according to a second embodiment of the present disclosure.

### DESCRIPTION OF EMBODIMENTS

Hereinafter, preferred embodiments of the present disclosure will be described in detail with reference to the appended drawings. It should be noted that, in this specification and the appended drawings, structural elements that have substantially the same function and structure are denoted with the same reference numerals, and repeated explanation of these structural elements is omitted.

Description will be provided in the following order.
1. First embodiment (When a processor unit retains display setting information)
1.1 System configuration
1.2 Display setting method
(1) Overview
(2) Functional configuration
(3) Display setting process
2. Second embodiment (When an IC card retains display setting information)
3. Conclusion

### 1. First Embodiment

### (1.1 System Configuration)

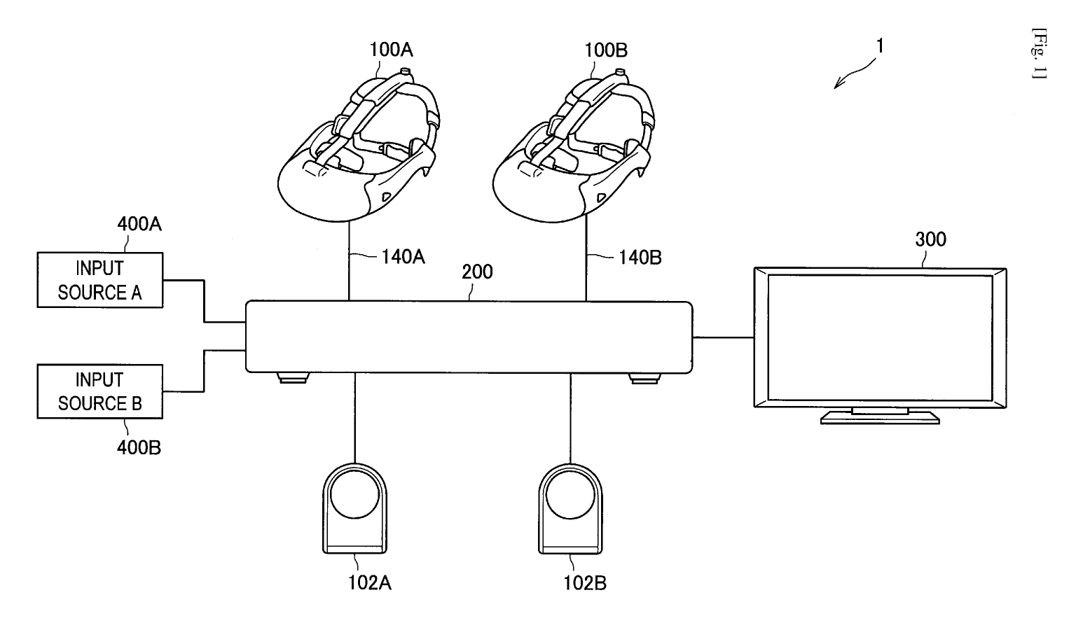

First, an endoscope system will be described as an example of a system in which an HMD according to a first embodiment of the present disclosure is used. FIG. 1 is a system configuration diagram illustrating a configuration example of the endoscope system 1 according to the present embodiment.

The endoscope system 1 according to the present embodiment is a system used in endoscopy operations, and an operator wears an HMD and conducts surgery while visually recognizing the state of an affected site captured by an endoscope device. The endoscope system 1 includes HMDs 100 (100A and 100B), a display 300, and external devices 400 (400A and 400B), all of which are connected with a processor unit 200, as illustrated in FIG. 1.

The HMDs 100 are display devices on which information from the external devices 400 such as an input video is displayed. The HMDs 100 are, for example, goggle-shaped non-transmissive HMDs, and users use them while wearing them on their heads. Each HMD 100 is composed of a main body part which has a display unit for presenting information to the wearer of the HMD 100, and an upper fixing part and a rear fixing part for fixing the main body part to the head. When the fixing parts are fixed to the head of the wearer, the display unit of the main body part is positioned in front of the left and right eyes of the wearer.

The main body part is a portion covering both eyes of the wearer. The main body part may be configured to cover, for example, near the left and right temples of the wearer. The shape described above enables the fronts of the eyes of the wearer to be covered substantially completely, and thus images can be easily seen without external light being incident on the eyes of the wearer. The main body part may have, for example, an imaging unit for photographing a peripheral environment on its outer surface. Accordingly, the wearer of the HMD 100 can also recognize information of a peripheral environment seen when he or she is not wearing the HMD 100 (video see-through) in addition to the information provided from the external devices 400 or the like via the processor unit 200.

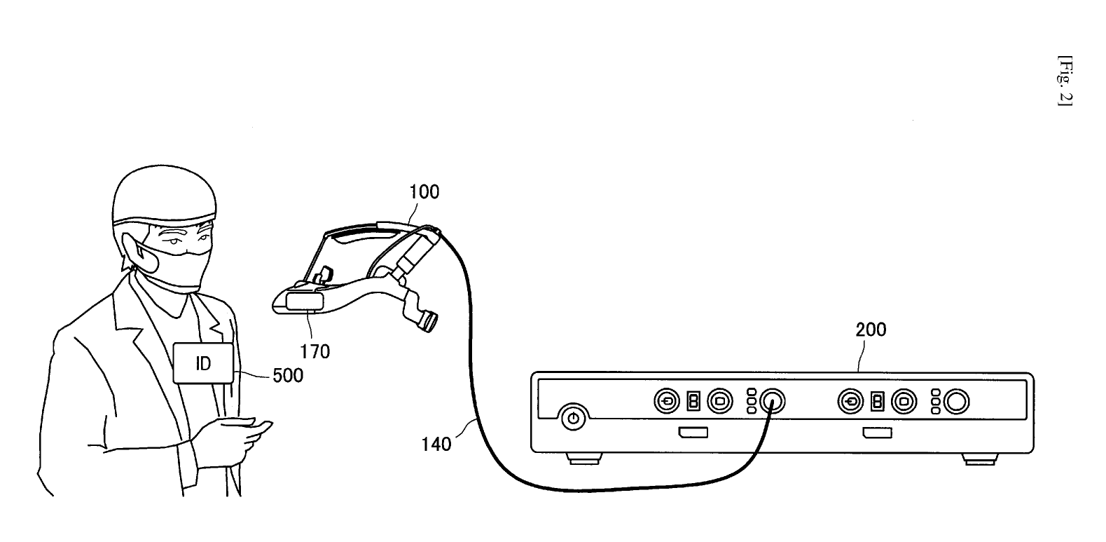

In addition, the HMD 100 according to the present embodiment is provided with a reader unit (reference numeral 170 of FIG. 2) for reading user identification information that is information unique to a user in, for example, the main body part. The reader unit is configured to be capable of acquiring information through, for example, near field communication (NFC) from an NFC-compliant device.

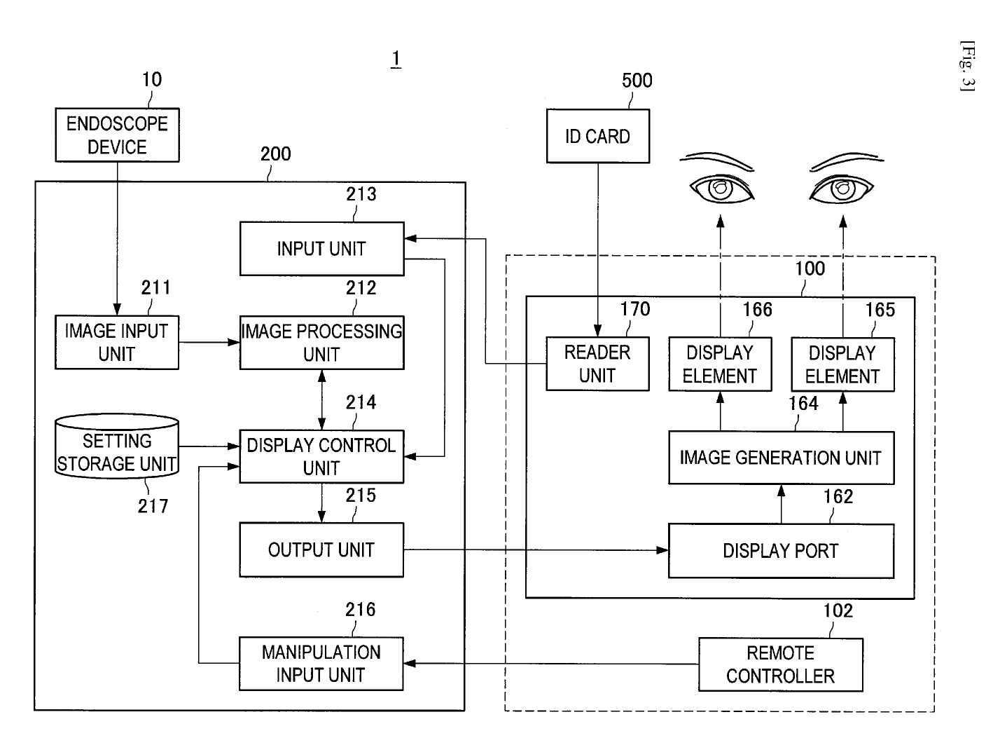

Inside the main body part, a first display element (reference numeral 165 of FIG. 3) which presents left-eye images to a first display unit and a second display element (reference numeral 166 of FIG. 3) which presents right-eye images to a second display unit are provided. Each of the display elements presents, for example, images of the endoscope device provided from the processor unit 200, images captured by the imaging unit of the main body part, and the like. It should be noted that a display control process of images displayed in the display unit of the HMD 100 will be described later. In addition, the main body part is provided with cables 140 (140A and 140B) connected to the processor 200 to perform transmission and reception of information with the processor unit 200. Although the HMDs 100 and the processor unit 200 are connected with wires in the present embodiment, an embodiment of the present disclosure is not limited thereto, and information communication between devices may be performed through wireless communication.

Information displayed in the display unit of the HMD 100 may be switched by remote controllers 102 (102A and 102B). The remote controllers 102 are provided to be paired with the respective HMDs 100. For example, the remote controllers may be foot switches with which the wearer performs stepping input manipulations using his or her foot. Input information from the remote controller 102 is output to the processor unit 200.

The processor unit 200 is a control device which controls connected devices. In the present embodiment, the processor unit 200 controls the HMDs 100 (100A and 100B), the display 300, and the external device 400 (400A and 400B) as illustrated in FIG. 1. Specifically, the processor unit 200 processes information input from the external devices 400 into information which can be displayed on the display units of the HMD 100 and the display 300, and outputs the information to each display device. In addition, the processor unit 200 switches information to be displayed on the display units of the HMDs 100 based on manipulation inputs from the remote controllers 102 of the respective HMDs 100.

The display 300 is an external display device for unspecified users to see information. The display 300 is mainly used by non-wearers of the HMDs 100 who work with the wearers of the HMDs 100 to see information. Input information from the external devices 400 and other information can be displayed on the display 300. Information to be displayed on the display 300 is set by the wearers, non-wearers, or the processor unit 200.

The external devices 400 are devices which output information to be displayed in the

HMDs 100 or display devices such as the display 300. In the endoscope system 1 of the present embodiment, for example, the external device A 400A is an endoscope device, and videos captured by a camera of the endoscope device are output to the processor unit 200.

Information input from the external devices 400 is processed by the processor unit 200 in the endoscope system 1 described above, and displayed on the HMDs 100 or a display device such as the display 300.

### (1.2 Display Setting Method)

Next, a display setting method of each HMD 100 according to the present embodiment will be described based on FIGS. 2 to 10.

### (1) Overview

First, an overview of the display setting method of the HMD 100 according to the present embodiment will be described based on FIG. 2. FIG. 2 is an illustrative diagram for describing an operation of a user during a display setting of the HMD 100 according to the present embodiment.

In the present embodiment, a display setting of the display unit of the HMD 100 is performed when the reader unit 170 provided in the HMD 100 to acquire information acquires user identification information. The user identification information is information unique to a user such as a user ID, and is acquired from an ID card 500 possessed by a user as illustrated in, for example, FIG. 2. The ID card 500 is an NFC-compliant card that stores a user ID, a user name, affiliation (department) of the user, and the like. When the ID card 500 is brought close to the reader unit 170 of the HMD 100, the reader unit 170 can read user identification information stored in the ID card 500. However, user identification may be performed without the ID card 500 in other embodiments. For example, user identification may be performed by using biological information of the user detected by a bio-sensor, such as iris or retina pattern recognition using a camera mounted on the HMD 100.

In the field of medicine, there are many cases in which it is prohibited to touch the HMD 100 using hands for hygienic reasons. Thus, using a non-contact IC card such as an NFC-compliant card, users can cause the reader unit 170 of the HMD 100 to read user identification information without using their hands even when, for example, the ID card 500 is placed underneath an operating gown. It should be noted that user identification information may be acquired from NFC-compliant devices and the like as well as from the ID card 500.

In addition, the user identification information acquired from the ID card 500 can also be used in determining an attribute of the user. For example, the user identification information is assumed to be associated with an attribute which indicates whether the user is a medical staff. In this case, it is possible to set the reader unit 170 such that, when the reader unit 170 reads the user identification information, the user of the user identification information is determined as a medical staff, and the HMD 100 is allowed to be used only when the user is a medical staff.

The user identification information acquired by the reader unit 170 of the HMD 100 is input to the processor unit 200 via the cable 140. The processor unit 200 stores display setting information of the display unit of the HMD 100 in association with the user identification information. The processor unit 200 acquires the display setting information which corresponds to the user identification information input from the HMD 100, and performs a display setting of the HMD 100 based on the acquired display setting information. Accordingly, images can be displayed with the display setting information set by the user in advance even when the user uses different HMDs 100 each time. In addition, when it is desired to set display settings of HMDs 100 of a plurality of users to be the same, the display settings of the HMDs 100 of the plurality of users can be the same only by causing the ID card 500 of a user which is set in a desired display setting to be read by the reader units 170 of the HMDs 100 used by the other users.

As described above, each of users can easily perform a display setting of the display unit of the HMD 100 in the display setting method of the HMD 100 according to the present embodiment, and thus it is not necessary to fix an HMD 100 to be used by a user. In addition, it is possible to save effort for settings even when display settings of a plurality of users are the same without necessitating a special manipulation.

### (2) Functional Configuration

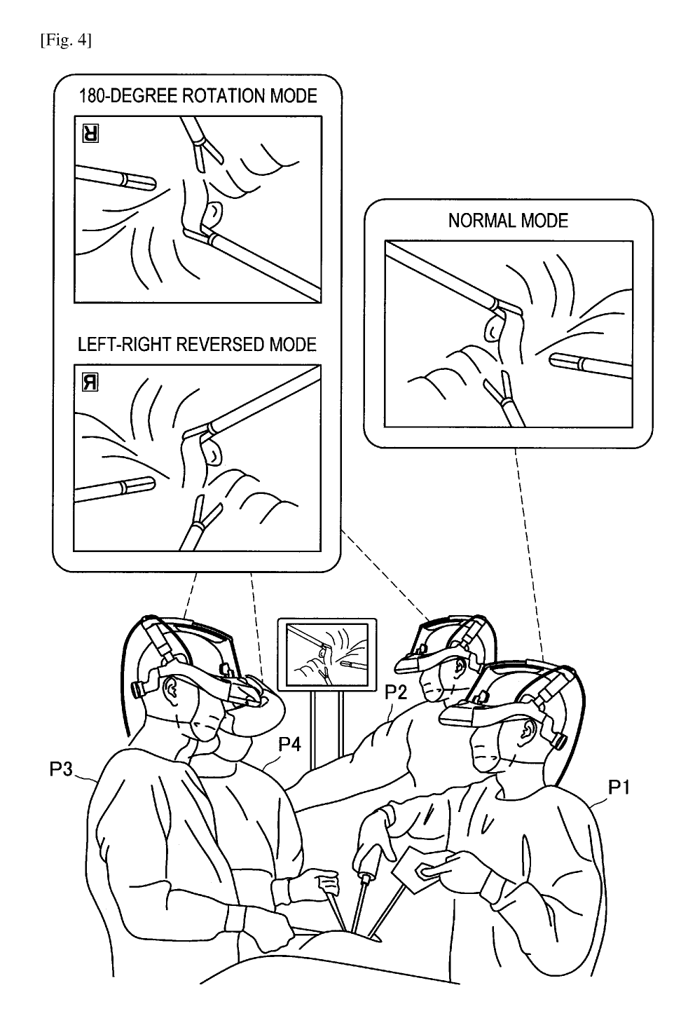

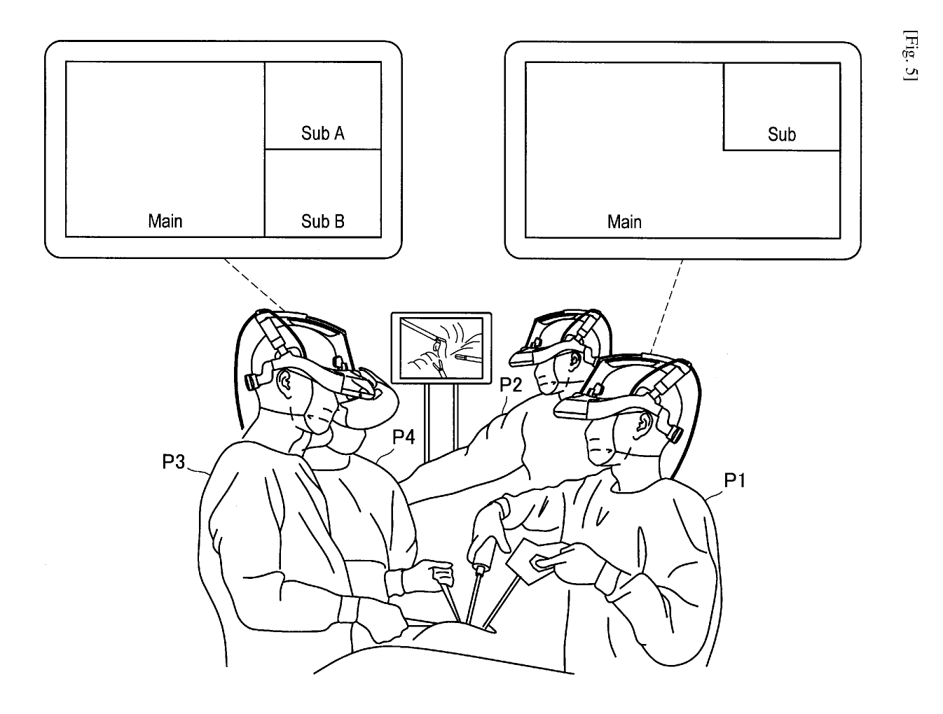

Next, a functional configuration of the HMD 100 and the processor unit 200 which constitute a display control system according to the present embodiment will be described with reference to FIGS. 3 to 5. FIG. 3 is a functional block diagram illustrating the functional configuration of the HMD 100 and the processor unit 200 which constitute the display control system according to the present embodiment. FIG. 4 is an illustrative diagram for describing a direction of display of a video that is one type of display setting information of the HMD 100. FIG. 5 is an illustrative diagram for describing disposition of videos which is one type of the display setting information of the HMD 100.

It should be noted that FIG. 3 illustrates functional units which function when display control of the display unit of the HMD 100 is performed, and actually, it is assumed to have other functional units. The processor unit 200 functions as a display control device which performs display control of the HMD 100 based on the display setting information associated with the user identification information acquired from the ID card 500.

First, in terms of a display processing function of the HMD 100, the HMD 100 has a display port 162, an image generation unit 164, the display elements 165 and 166, and the reader unit 170 as illustrated in FIG. 3.

The display port 162 is an interface which receives input information from the processor unit 200. The display port 162 is connected with the cables 140 which enable information communication with the processor unit 200. The display port 162 receives inputs of, for example, image signals each output to the display elements 165 and 166, and information that the wearer of the HMD 100 visually recognizes. Information input from the display port 162 is output to the image generation unit 164.

The image generation unit 164 generates image signals to be output to each of the display elements 165 and 166 based on information acquired through the processor unit 200. When an image to be presented to the wearer is a 3D image, the image generation unit 164 performs a shifting process of causing a deviation to occur between a left-eye image signal to be output to the first display element 165 and a right-eye image signal to be output to the second display element 166. In the shifting process, an amount of shifting between the left-eye signal and the right-eye signal is decided according to, for example, the distance between the display elements 165 and 166 and the eyes of the wearer, the gap between the eyes of the wearer, a position of a virtual image, and the like. The image generation unit 164 outputs the generated image signals to the first display element 165 and the second display element 166.

The display elements 165 and 166 emit image light toward the display unit based on the image signals input from the image generation unit 164. The display elements 165 and 166 are disposed, for example, to face the display unit in the front-rear direction of the face of the wearer while he or she wears the HMD 100. Accordingly, the optical axis of the image light emitted from the display elements 165 and 166 becomes substantially parallel with the direction of a line of sight when the wearer faces the front direction.

The display elements 165 and 166 are configured by, for example, organic electro-luminescence (EL) elements. Adoption of organic EL elements as the display elements 165 and 166 realizes a small size, high contrast, quick responsiveness, and the like. The display elements 165 and 166 have a configuration in which, for example, a plurality of red organic EL elements, green organic EL elements, and blue organic EL elements are disposed in a matrix shape. Each of the elements is driven by an active matrix-type or a passive matrix-type drive circuit, and thereby emits light by itself at a predetermined time point, with predetermined luminance, and the like. As the drive circuit is controlled based on the image signals generated by the image generation unit 164, a predetermined whole image is displayed by the display elements 165 and 166, and the image is provided to the wearer via the display unit.

It should be noted that, for example, a plurality of ocular lenses (not illustrated) may be disposed between the display elements 165 and 166 and the display unit as an optical system. By setting the ocular lenses to face the eyes of the wearer at a predetermined distance, the wearer can observe a virtual image as if it were displayed at a predetermined position (a virtual image position). With presentation of the virtual image, a 3D image can be provided. It should be noted that the virtual image position and the size of a virtual image can be set according to a configuration of the display elements 165 and 166 and the optical system or the like.

The reader unit 170 is a device which reads user identification information from the

ID card 500. The reader unit 170 is provided on an outer surface of the main body part as illustrated in, for example, FIG. 2. The reader unit 170 acquires user identification information from the ID card 500 or the like in proximity to the reader unit 170 at a predetermined distance or closer through NFC, and transmits the information to the processor unit 200.

Next, the display processing function of the processor unit 200 will be described. The processor unit 200 has an image input unit 211, an image processing unit 212, an input unit 213, a display control unit 214, an output unit 215, a manipulation input unit 216, and a setting storage unit 217 as illustrated in FIG. 3.

The image input unit 211 is an interface which receives images input from the external devices 400 to the processor unit 200. In the example of FIG. 3, an endoscope device 10 is illustrated as the external device 400, and images captured by a camera (not illustrated) of the endoscope device 10 are input to the image input unit 211 in this case. The image input unit 211 outputs the input images to the image processing unit 212.

The image processing unit 212 processes images input to the processor unit 200 as images to be displayed in the HMD 100. The image processing unit 212 generates left-eye images to be displayed on the first display unit and right-eye images to be displayed on the second display unit of the HMD 100 from the images captured by the camera of the endoscope device 10. The images processed by the image processing unit 212 are output to the display control unit 214.

The input unit 213 is an interface to which the user identification information acquired by the reader unit 170 of the HMD 100 is input. The user identification information input to the input unit 213 is output to the display control unit 214.

The display control unit 214 controls information to be displayed on the display unit of the HMD 100. The display control unit 214 controls information instructed to be displayed based on a display switch instruction from the remote controllers 102. In addition, the display control unit 214 acquires corresponding display setting information based on the user identification information input from the input unit 213, and performs a display setting based on the acquired display setting information. When the information to be displayed in each HMD 100 and display setting thereof are decided, the display control unit 214 outputs the information to each HMD 100 via the output unit 215.

The manipulation input unit 216 is an input unit which receives manipulation inputs from the wearer of the HMD 100. In the present embodiment, the information to be displayed on the display unit of the HMD 100 can be switched by the remote controllers 102. Manipulation inputs of the remote controllers 102 are output to the manipulation input unit 216, and the manipulation input unit 216 outputs the manipulation input information to the display control unit 214. The display control unit 214 outputs information to the HMD 100 as instructed via the output unit 215 based on a display switch instruction from the remote controller 102.

The setting storage unit 217 is a storage unit which stores the display setting information of the HMD 100 which corresponds to each piece of user identification information. The display setting information stored in the setting storage unit 217 includes various kinds of setting information, for example, image quality, directions of images, disposition of images, and the like. The setting information with respect to image quality is information which represents a value of setting of, for example, brightness, tint, or the like of an image. Information with respect to a direction of an image is information which represents a display direction of the image to be displayed on the display unit. Here, the display direction of an image indicates a change in the display state of a reference image.

For example, in a situation in which 4 users P1 to P4 are working, the display units of the HMDs 100 worn by the respective users P1 to P4 each display an image photographed by the camera manipulated by the user P1 as illustrated in FIG. 4. The display unit of the HMD 100 worn by the user P1 displays the image of a normal mode illustrated on the right side of FIG. 4. This image of the normal mode serves as a reference. Here, when a photographing target is surrounded by the users P1 to P4, views of the photographing target are different according to the standing positions of the users. For example, for the users P3 and P4 who are positioned to face the user P1 having the photographing target therebetween, displaying the image rotated 180 degrees from its normal mode is close to the actual views of the users. In addition, for the user P2 who is positioned beside the user P1, it may be better to display the image by turning the normal mode into left-right reversed mode.

The standing positions of the respective users P1 to P4 are mostly decided according to their roles during the work. Thus, unless the information is setting information that is changed very often, it is possible to lower burdens of display settings on the users by storing the setting with respect to the display direction of an image in advance in association with user identification information of the users.

In addition, information of disposition of images is information which represents, when one or more images can be displayed in a display region at the same time using a PIP function, which image is to be displayed in what kind of disposition. For example, in the example shown in FIG. 5, the user P1 is setting a main screen to be displayed over the entire display unit of the HMD 100 and a sub screen to be displayed in a small size on the upper right side of the main screen. For example, if a video of the endoscopic camera is set as a main image and a CT-scanned image is set as a sub image, the video of the endoscopic camera is displayed on the main screen in a large size and the CT-scanned image is displayed on the upper right side thereof.

In addition, the user P3 is setting a main screen on the left side of the display unit of the HMD 100 and two sub screens to be displayed on the right side of the main screen. For example, if a video of the endoscopic camera is set as a main image and a radiographic picture and an outer field of view image (video see-through image) is set as sub images, the video of the endoscopic camera is displayed on the main screen and the radiographic picture and the video see-through image are displayed on the right side of the main screen being arranged up and down. In this manner, images that respective users want to see can be presented in user-friendly dispositions.

The setting storage unit 217 stores the display setting information of the images to be displayed on the display unit of the HMD 100 as above in association with the user identification information. Note that the display setting information stored in the setting storage unit 217 may be set to store changed settings when the users have changed settings.

### (3) Display Setting Process

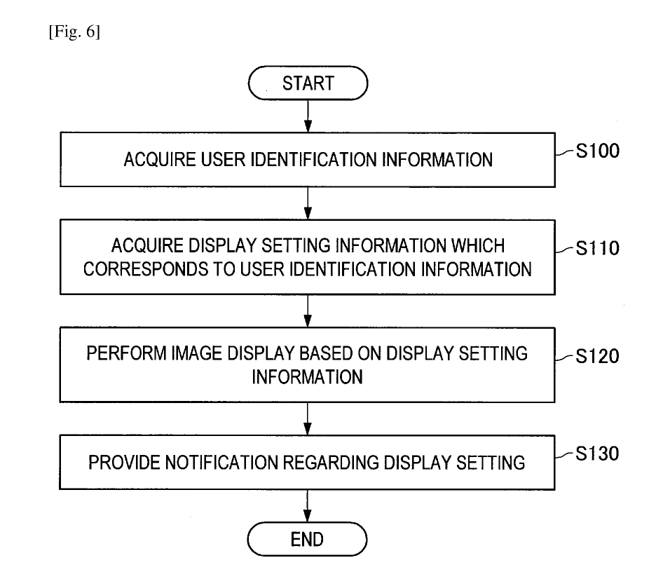

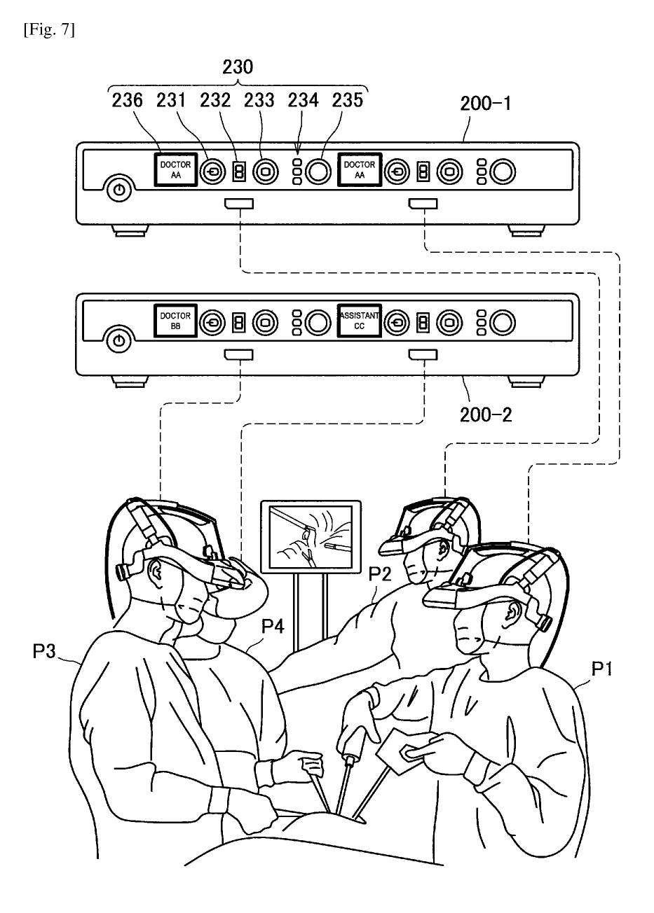

A display process based on the display setting information of the display control system according to the present embodiment will be described based on FIGS. 6 to 9. FIG. 6 is a flow chart showing the display process based on the display setting information of the display control system according to the present embodiment. FIGS. 7 to 9 are illustrative diagrams illustrating examples in which states of display settings are displayed on the processor unit 200.

In order to perform a display setting of the display unit of the HMD 100 in the display control system according to the present embodiment, first, the reader unit 170 of the HMD 100 to be set is caused to acquire user identification information (S100). A user causes the reader unit 170 of the HMD 100 to acquire user identification information by bringing the ID card 500 retaining the user identification information close to the reader unit as illustrated in, for example, FIG. 2. The user identification information acquired by the reader unit 170 is output to the processor unit 200 via the cable 140.

Next, the processor unit 200 which has received an input of the user identification information from the HMD 100 acquires display setting information which corresponds to the user identification information from the setting storage unit 217 for the display control unit 214 (S110). Upon acquiring the display setting information associated with the user identification information, the display control unit 214 then controls an image processed by the image processing unit 212 to be displayed in the HMD 100 based on the display setting information (S120). The display control unit 214 sets, for example, brightness or tint of the image, a display direction of the image, the number of images to be displayed, disposition of the images, and the like based on the content set in the display setting information. It should be noted that, for display setting information that is not designated in the display setting information, a predetermined value set in advance is set.

When the image is set based on the display setting information, the display control unit 214 outputs image data to the HMD 100 via the output unit 215. At this time, the display control unit 214 may cause a notification unit provided in the processor unit 200 to display the user identification information on which the display setting of the HMD 100 is based (S130).

The processor unit 200 is provided with various notification units which indicate setting states of the HMD 100 connected to the processor unit 200 and various manipulation buttons for manipulating the HMD 100. For example, processor units 200-1 and 200-2, each of which is connected to two HMDs 100, are provided with manipulation notification units 230 for the HMDs 100 as illustrated in FIG. 7.

In the manipulation notification unit 230, there are, for example, an input signal selection button 231 for images to be output to the HMD 100, an input image notification button 232 for providing notifications regarding images to be output to the HMD 100, and a PIP button 233 for switching display of a sub screen on or off as illustrated in FIG. 7. Furthermore, in the manipulation notification unit 230, there are also a reversed display indicator 234 for indicating a direction of an image being displayed, a reversed display switch button 235 for switching a direction of an image being displayed, and the like. Moreover, in the manipulation notification unit 230, there may be a setting notification unit 236 which provides a notification regarding the user (user identification information) on which a display setting is based when the setting of the display unit of the HMD 100 is automatically performed based on the user identification information as described in the present embodiment.

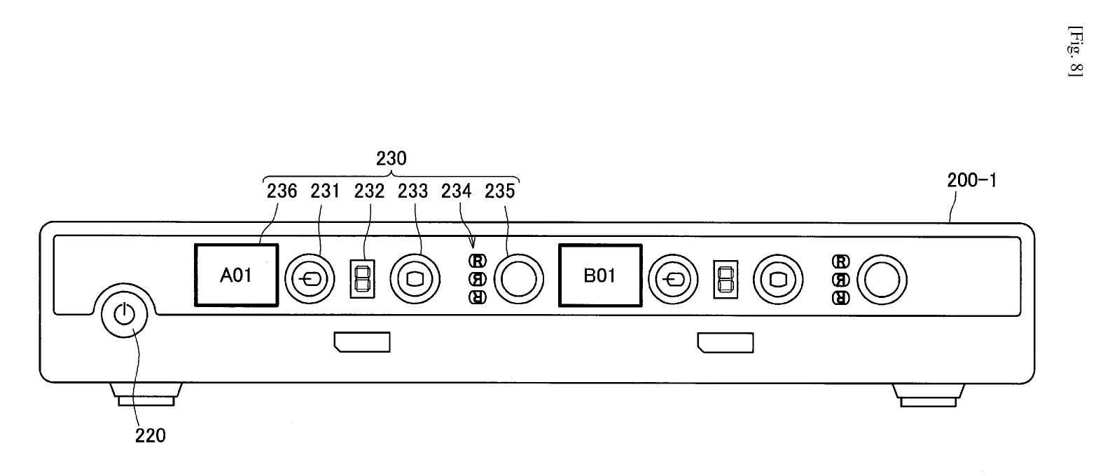

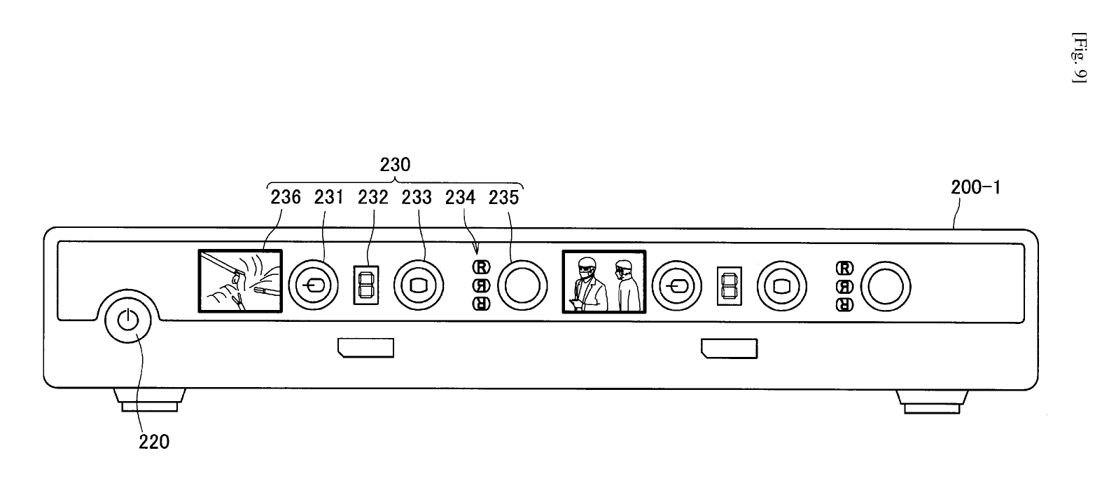

The setting notification unit 236 can be configured as, for example, a display panel on which information can be displayed or the like. As the content displayed on the setting notification unit 236, for example, a user name (for example, “Doctor AA” or the like) that has the user identification information associated with the display setting information may be displayed as illustrated in FIG. 7. Alternatively, a user ID (“A01,” “B01,” or the like) that is the user identification information may be displayed on the setting notification unit 236 as illustrated in FIG. 8, or an image being displayed in the HMD 100 may be displayed on the setting notification unit 236 as illustrated in FIG. 9.

By displaying the state of a display setting of the HMD 100 in the processor unit 200 as described above, when, for example, a third party changes an image displayed in the HMD 100, it is possible to prevent erroneous manipulations in which a display setting of another HMD 100 is mistakenly changed. Furthermore, the content displayed on the setting notification unit 236 of the processor unit 200 may also be displayed in the HMD 100 in which the setting has been made. Accordingly, a person near the user who is wearing the HMD 100 can more reliably recognize the display setting of each HMD 100.

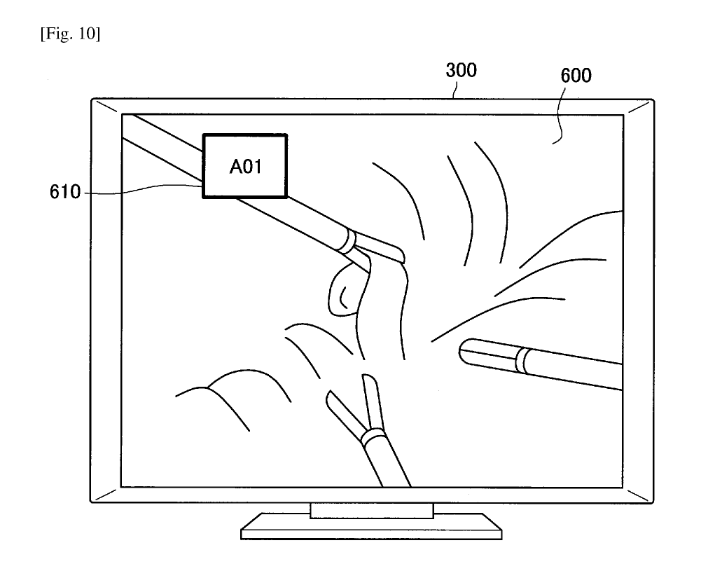

In addition, the processor unit 200 may cause the state of a display setting of each HMD 100 to be displayed on the external display 300. For example, an image 600 being displayed in the HMD 100 of a certain user and a notification object 610 which represents user identification information of the user who is using the HMD 100 may be displayed on the display 300 as illustrated in FIG. 10. In the notification object 610, a user ID, a user name, or the like is displayed as illustrated in FIG. 10. Accordingly, people other than the wearer of each HMD 100 can be notified of the user identification information on which the display setting of the HMD 100 is based.

In addition, when the display plane of the external display 300 is configured as a touch panel or the like, a display setting displayed on the display 300 can be configured to be adjustable by manipulating an object which indicates a state of the setting. For example, the display setting of the HMD 100 may be changed all at once or various setting states included in the display setting information may be changed by changing a user ID that is one kind of the user identification information. Accordingly, people other than the wearer of the HMD 100, for example, a nurse and the like, can also perform a manipulation of switching the display setting of the HMD 100 or the like with ease.

The display setting process of the display control system according to the present embodiment has been described above. According to the present embodiment, display setting information set in advance is acquired based on user identification information acquired by the reader unit 170 of the HMD 100 from the ID card 500 or the like, and thereby the display setting of the HMD 100 is performed. Accordingly, users can also easily perform the display setting of the HMD 100 to be used without fixing the HMD 100 to be used. In addition, the display setting of a certain user can be easily shared with a plurality of users.

### 2. Second Embodiment

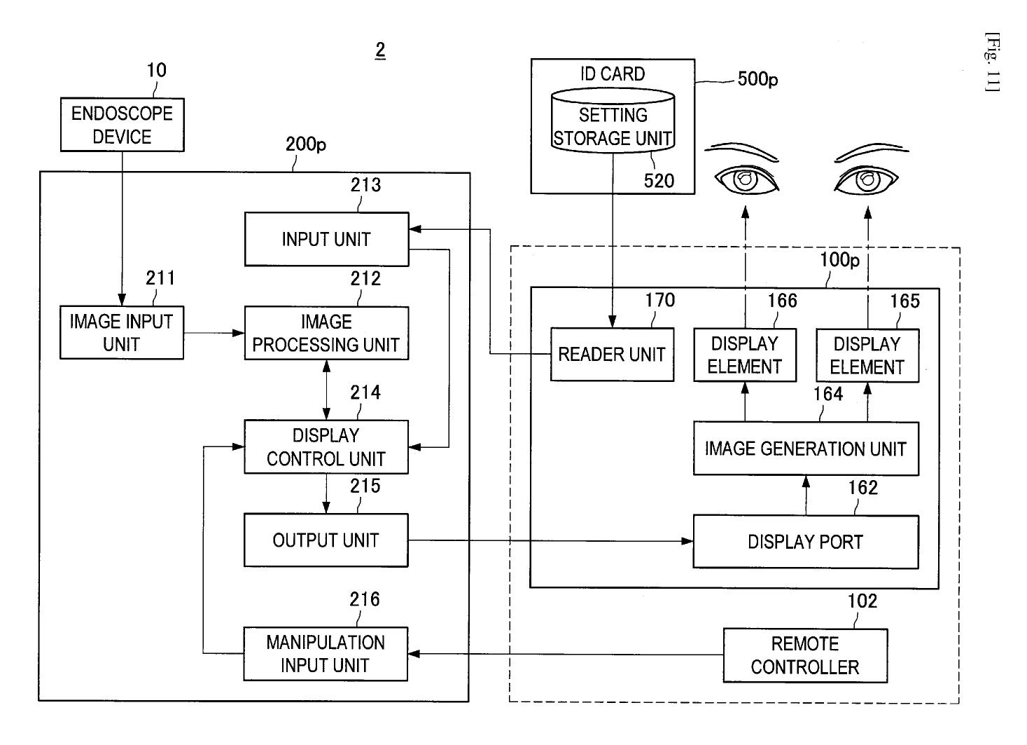

A display control system according to a second embodiment of the present disclosure will be described based on FIG. 11. FIG. 11 is a functional block diagram illustrating a functional configuration of an HMD and a processor unit constituting the display control system according to the present embodiment. The case in which an endoscope system 2 is applied to the display control system according to the present embodiment as in the first embodiment will be described herein. The display control system according to the present embodiment is different from the display control system of the first embodiment in that display setting information of an HMD 100p of each user is stored in an ID card 500p. The difference from the first embodiment will be described below, and detailed description in regard to the same functional units as those of the first embodiment will be omitted.

First, in terms of a display processing function of the HMD 100p, the HMD 100p has a display port 162, an image generation unit 164, the display elements 165 and 166, and the reader unit 170 as illustrated in FIG. 11. This functional configuration is the same as that of the HMD 100 of the first embodiment.

The reader unit 170 also acquires display setting information of the HMD 100p in addition to user identification information from the ID card 500p in the present embodiment. The ID card 500p includes a memory that stores a setting storage unit 520, and the setting storage unit 520 is assumed to store the display setting information of the HMD 100p set by each user in advance. The reader unit 170 transmits the acquired user identification information and display setting information to a processor unit 200p.

On the other hand, for the display processing function of the processor unit 200p, the processor unit 200 includes the image input unit 211, the image processing unit 212, the input unit 213, the display control unit 214, the output unit 215, and the manipulation input unit 216 as illustrated in FIG. 11. This functional configuration is the same as that of the processor unit 200 of the first embodiment. It should be noted that the processor unit 200p of the present embodiment may not be provided with a setting storage unit which stores the display setting information.

The input unit 213 of the present embodiment is an interface which receives inputs of the user identification information and display setting information acquired by the reader unit 170 of the HMD 100p. The information input to the input unit 213 is output to the display control unit 214.

The display control unit 214 controls information to be displayed on the display unit of the HMD 100p. The display control unit 214 controls information to be displayed as instructed based on a display switch instruction from the remote controller 102. In addition, the display control unit 214 performs a display setting of the HMD 100p based on the display setting information input from the input unit 213, and outputs an image input from the image processing unit 212 to each HMD 100p via the output unit 215.

As described above, by retaining the display setting information of the HMD 100p together with the user identification information, it is not necessary to retain the display setting information of each user in the processor unit 200p. Thus, an image can be displayed in the HMD 100p under a desired display setting of the user even for the HMD 100p and the processor unit 200p to be used by a user for the first time. The state of a display setting of the HMD 100p may be displayed in the HMD 100p or the external display 300 as in the first embodiment.

### 3. Conclusion

The configurations of the display control system and the display setting processes based on the configurations according to the embodiments of the present disclosure have been described so far. According to the above-described embodiments, the reader unit 170 of the HMD 100 acquires at least the user identification information for specifying a user. Then, based on the display setting information of the HMD 100 associated with the user identification information, a display setting of the HMD 100 which has acquired the user identification information is performed. Thereby, the user can easily perform a desired display setting without fixing his or her HMD 100 to be used. In addition, since the display setting of the HMD 100 can be performed by holding the ID card 500 over the reader unit 170 of the HMD 100, display setting information of a certain user can also be easily shared by a plurality of users.

Further, although the present embodiments have been discussed with the HMD 100, it is noted that one or a combination of other wearable display devices, such as eyeglasses, near-eye display, or contact lens type displays may be used with or as an alternative to the HMD 100. It should also be understood that the HMD 100 and other wearable display devices, as well as the embodiments of the present disclosure, are not limited to medical uses and are applicable to gaming or other displaying systems in other embodiments.

In addition, although the cases in which the proposed display control system is applied to the endoscope system have been described in the above-described embodiments, an embodiment of the present disclosure is not limited thereto. For example, an application of the display control system to a display setting of an in-vivo image acquired using medical devices other than an endoscope is considered. When the HMD is used in observation of blood vessels in angiography, a therapeutic treatment performed in that case, or the like, or when an optical microscopic image is viewed using the HMD in a cerebral surgical operation, for example, display settings thereof may be performed with the display control system. Furthermore, when an ultrasonic image and another image are viewed at the same time using the HMD during work performed in a dark place of an ultrasonic inspection or the like or when the HMD is used during a laparotomy in which a magnifier is used, the display setting of the HMD can also be performed with the above-described display control system.

It should be understood by those skilled in the art that various modifications, combinations, sub-combinations and alterations may occur depending on design requirements and other factors insofar as they are within the scope of the appended claims or the equivalents thereof.

Although the reader unit 170 which reads user identification information is set to acquire, for example, the information through NFC in the above-described embodiments, an embodiment of the present technology is not limited thereto. It may be set as a reader unit which acquires, for example, biological information of the wearer of the HMD 100 as the user identification information. In this case, the user identification information may be, for example, the iris of an eye, a fingerprint, or the like.

In addition, the effects described in the present specification are merely illustrative and demonstrative, and not limitative. In other words, the technology according to the present disclosure can exhibit other effects that are evident to those skilled in the art along with or instead of the effects based on the present specification.

Additionally, the present technology may also be configured as below.

(1)

A surgical system, including:
a surgical imaging device configured to capture a surgical image;
a plurality of head-mounted displays; and
circuitry configured to
receive user identification information from each of the plurality of head-mounted displays, determine a display setting for each of the plurality head-mounted displays based on display setting information associated with the user identification information received from the respective one of the plurality of head-mounted displays, and
set the display settings of the plurality of head-mounted displays based on the determined display settings to display the surgical image from the surgical imaging device.

(2)

The surgical system according to (1), wherein the surgical imaging device includes an endoscope or a microscope.

(3)

The surgical system according to claim (1) or (2), wherein each of the plurality of head-mounted displays includes a reader configured to acquire the user identification information.

(4)

A surgical display system, comprising:
circuitry configured to
receive user identification information,
determine a display setting for each of a plurality of head-mounted displays based on display setting information associated with the received user identification information, and
set the display settings of the plurality of head-mounted displays based on the determined display settings to display an image from a surgical imaging device.

(5)

The surgical display system according to (4), further including
a memory configured to store the display setting information,
wherein the circuitry is configured to receive the user identification information acquired by readers of the plurality of head-mounted displays for which the display settings are to be set, and
wherein the circuitry acquires the display setting information associated with the received user identification information from the memory.

(6)

The surgical display system according to (4) or (5), in which the circuitry is configured to
receive the user identification information and the display setting information acquired by readers of the plurality of head-mounted displays for which the display settings are to be set, and
set the display setting of each of the plurality of head-mounted displays based on the display setting information received from the respective one of the plurality of head-mounted displays.

(7)

The surgical display system according to any one of (4) to (6), in which the circuitry is further configured to provide a notification regarding a state of the display setting of each of the plurality of head-mounted displays.

(8)

The surgical display system according to any one of (4) to (7), wherein the circuitry causes a state of the display setting for each of the head-mounted displays to be displayed on an external display device.

(9)

The surgical display system according to any one of (4) to (8), wherein the circuitry causes the user identification information of each of the head-mounted displays to be displayed on an external display device.

(10)

The surgical display system according to any one of (4) to (9), wherein the display setting information is at least one of image quality, disposition of images, and a display direction of the image.

(11)

The surgical display system according to any one of (4) to (10), wherein the user identification information includes information unique to a user stored in a non-contact IC card.

(12)

The surgical display system according to any one of (4) to (11), wherein the display setting information is related to display of an ultrasonic image or an angiography image for each of the plurality of head-mounted displays.

(13)

The surgical display system according to any one of (4) to (12), wherein the surgical imaging device is an endoscope or a microscope.

(14)

A method of a surgical display system for controlling display of an image, including:
receiving user identification information;
determining, by circuitry of the surgical display system, a display setting for each of a plurality of head-mounted displays based on display setting information associated with the received user identification information; and
setting, by the circuitry, the display settings of the plurality of head-mounted displays based on the determined display settings.

(15)

A head-mounted display including:
circuitry configured to
acquire at least user identification information; and
output the acquired user identification information to a display control device.

### REFERENCE SIGNS LIST

1 endoscope system
100 HMD
102 remote controller
162 display port
164 image generation unit
165 first display element
166 second display element
170 reader unit
200 processor unit
211 image input unit
212 image processing unit
213 input unit
214 display control unit
215 output unit
216 manipulation input unit
217 setting storage unit (processor unit)
300 display
400 external device
500 ID card
520 setting storage unit (ID card)

1. A surgical system, comprising:
a surgical imaging device configured to capture a surgical image;
a plurality of head-mounted displays; and
circuitry configured to
receive user identification information from each of the plurality of head-mounted displays,
determine a display setting for each of the plurality head-mounted displays based on display setting information associated with the user identification information received from the respective one of the plurality of head-mounted displays, and
set the display settings of the plurality of head-mounted displays based on the determined display settings to display the surgical image from the surgical imaging device.

2. The surgical system according to claim 1, wherein the surgical imaging device includes an endoscope or a microscope.

3. The surgical system according to claim 1, wherein each of the plurality of head-mounted displays includes a reader configured to acquire the user identification information.

4. A surgical display system, comprising:
circuitry configured to
receive user identification information,
determine a display setting for each of a plurality of head-mounted displays based on display setting information associated with the received user identification information, and
set the display settings of the plurality of head-mounted displays based on the determined display settings to display an image from a surgical imaging device.

5. The surgical display system according to claim 4, further comprising:
a memory configured to store the display setting information,
wherein the circuitry is configured to receive the user identification information acquired by readers of the plurality of head-mounted displays for which the display settings are to be set, and
wherein the circuitry acquires the display setting information associated with the received user identification information from the memory.

6. The surgical display system according to claim 4, wherein the circuitry is configured to
receive the user identification information and the display setting information acquired by readers of the plurality of head-mounted displays for which the display settings are to be set, and
set the display setting of each of the plurality of head-mounted displays based on the display setting information received from the respective one of the plurality of head-mounted displays.

7. The surgical display system according to claim 4, wherein the circuitry is further configured to
provide a notification regarding a state of the display setting of each of the plurality of head-mounted displays.

8 The surgical display system according to claim 4, wherein the circuitry causes a state of the display setting for each of the head-mounted displays to be displayed on an external display device.

9. The surgical display system according to claim 4, wherein the circuitry causes the user identification information of each of the head-mounted displays to be displayed on an external display device.

10. The surgical display system according to claim 4, wherein the display setting information is at least one of image quality, disposition of images, and a display direction of the image.

11. The surgical display system according to claim 4, wherein the user identification information includes information unique to a user stored in a non-contact IC card.

12. The surgical display system according to claim 4, wherein the display setting information is related to display of an ultrasonic image or an angiography image for each of the plurality of head-mounted displays.

13. The surgical display system according to claim 4, wherein the surgical imaging device is an endoscope or a microscope.

14. A method of a surgical display system for controlling display of an image, comprising:
receiving user identification information;
determining, by circuitry of the surgical display system, a display setting for each of a plurality of head-mounted displays based on display setting information associated with the received user identification information; and
setting, by the circuitry, the display settings of the plurality of head-mounted displays based on the determined display settings.

15. A head-mounted display comprising:
circuitry configured to
acquire at least user identification information; and
output the acquired user identification information to a display control device.
